(19) United States
(12) Patent Application Publication
(10) Pub. No.: US 2005/0048007 A1
(43) Pub. Date: Mar. 3, 2005

(54) Plaque reducing composition

(75) Inventor: N. Garison Ruggles, Berkeley, CA (US)

Correspondence Address: BEYER WEAVER & THOMAS LLP, P.O. BOX 70250, OAKLAND, CA, 94612-0250, US

(73) Assignee: INOBYS LTD.

(21) Appl. No.: 10/962,070

(22) Filed: Oct. 7, 2004

Related U.S. Application Data
(63) Continuation of application No. 10/053,501, filed on Nov. 2, 2001, now abandoned.
(60) Provisional application No. 60/245,695, filed on Nov. 2, 2000.

(51) Int. Cl. A61K 35/78; A61K 7/16; A61K 7/26

(52) U.S. Cl. 424/58; 424/757

(57) ABSTRACT

Anti-odor and anti-plaque compositions for oral use are provided comprising an anti-odor and anti-plaque active component of an extract of Glycyrrhiza glabra or Usnea spp., an anti-odor or anti-plaque active component of such extracts, and mixtures thereof. The composition may further comprise a cationic and/or non-ionic surfactant, divalent metal cations, oligosaccharides, suitable solubilizing carriers and other additives.

### CROSS-REFERENCE TO RELATED APPLICATIONS

This application claims the benefit of Provisional Application No. 60/245,695, filed on Nov. 2, 2000, which is incorporated by reference herein.

### TECHNICAL FIELD

This invention relates to oral hygiene, and more particularly to antiplaque compositions.

### BACKGROUND

The present invention relates to oral compositions containing extracts or chemically synthesized compounds of the active ingredients of such extracts, of Glycyrrhiza glabra (commonly known as licorice) or Usnea spp. (commonly known as Usnea lichen). These compositions are useful for oral application for the reduction of plaque.

In the oral cavity a number of microbial species make up dental plaque. However, only certain of these microbes become pathogenic and are capable of initiating dental caries and periodontal disease when their numbers rise above a threshold level. Plaque-induced diseases, including periodontitis and gingivitis are apparently anaerobic bacterial infections. While gingivitis and periodontitis are inflammatory disorders, gingivosis and peridontosis are more severe conditions which involve degenerative conditions of the tissue. Therefore, an important feature of oral health is to continuously suppress the dental plaque, the proliferation of which can lead to these more severe conditions.

While good oral hygiene, such as that achieved by brushing and cleansing with a dental floss may help reduce the incidence of periodontal disease, it does not necessarily prevent or eliminate its occurrence because microorganisms contribute to both the initiation and progress of the disease. These microorganisms must be suppressed by some means other than simple mechanical scrubbing. This has resulted in research aimed at developing therapeutic dentifrices, mouthwashes and other methods of treating periodontal disease which are effective in suppressing the microorganisms.

The proliferation of certain microbial organisms which produce acids (acidogenic) are able to survive in acid environments (aciduric) and are prevalent in dental plaque. These species can produce sufficient acid to dissolve dentin which leads to fissure caries (cavities of molar teeth) and root caries (cavities located in exposed areas of dentin on the roots). The treatments of dental caries typically involve a combination of brushing or rinsing to remove bacteria plaque, contacting with agents which fortify the enamel, treatment with products which inhibit adherence of plaque to the teeth, pH altering agents, and antibacterial agents. Fluoride, in the form of fluorididated water, toothpaste and rinses has been an important caries preventing composition. It is believed to fortify the enamel and have antibacterial properties. However, since there is a need for continuous daily rinsing at reasonably high fluoride concentrations for it to be effective, there is a concern over the possibility of fluoride toxicity by such frequent use.

Although there have been a number of approaches disclosed for dealing with plaque and combating caries and periodontal disease, there is still the desire and need to develop improved products possessing properties which reduce or eliminate the occurrence or proliferation of these diseases.

### SUMMARY

The invention relates to compositions for anti-plaque, anti-cariogenic and anti-perodontolpathic therapy. The compositions comprise an anti-odor and anti-plaque active component which is an extract of Glycyrrhiza glabra, an extract of Usnea spp., a synthetic compound of an active ingredient of such extracts, or mixtures of these extracts or compounds. The composition contains at least about 0.0001% by weight of active ingredient. The composition may contain a cationic or nonionic surfactact and in aqueous form has a pH of, less than about 5. The composition may also contain divalent metal cations and oligosaccharides.

Plaque-forming bacteria are reduced by administering to the oral cavity an effective amount of a composition of the present invention administered as a mouthwash, rinse, mouth spray, gel, toothpaste, dental butter, buccoadhesive tablet or film, rapid dissolving tablet or film, chewable polymer or raw hide, or oral swab or wipe.

The details of one or more embodiments of the invention are set forth in the accompanying drawings and the description below. Other features, objects, and advantages of the invention will be apparent from the description and the claims.

### DETAILED DESCRIPTION OF PREFERRED EMBODIMENTS

As used in the following description, the terms have the following meanings.

Glycyrrhiza glabra extract, also known as licorice root extract, refers to powder isolated from extraction of Glycyrrhiza glabra varieties. Such varieties include G.tipica, G.inflata and G.glanduflora. The extracts include such constituents as glabrene, glabridin, licochalcone A, licochalcone B, glycyrrhizic acid and glycyrrhetinic acid or other flavonoids and coumarins. In preferred embodiments, the G.glabra extracts included in the compositions of the present invention may preferably include only water insoluble G.glabra extracts. These extracts (both water soluble and water insoluble) are available from commercial sources or by solvent extraction in the laboratory.

Usnea spp. extract refers to an extract of various species of lichen including U.hirta, U.barbata, U.florida, U.longissima, U.dasypoga, U.bayle, U.lobata, U.californica, Usnea barbata and florida are the most commonly used species. In preferred embodiments, the Usnea spp. extracts included in the compositions of the present invention may preferably include only water insoluble Usnea spp. extracts. These extracts (both water soluble and water insoluble) are available from commercial sources or by solvent extraction in the laboratory. In place of or in addition to the extracts, synthesized compounds which are active ingredients of the extracts may be used, such as glabrins, usnic acids or usnic acid salts. The primary active ingredients in the compositions of the present invention comprise the extracted or synthesized components from Glycyrrhiza glabra and Usnea spp. These components will be present in the composition in the concentration of at least about 0.0001% by weight.

The composition of the present invention may also contain a cationic surfactant and/or nonionic surfactant. The cationic surface active agents include, but are not limited to, alkylammonium compounds (including saturated or unsaturated heterocycles), alkenylamines and primary alkylamines, secondary or tertiary alkylamines, tertiary amines, amine ethers or primary, secondary or tertiary alkylene-diamines. Quaternary ammonium compounds are particularly useful, such as pyridinium and isoquinolinium compounds. Such compounds include, but are not limited to, cetyl pyridinium chloride, hexadecyl pyridinium chloride, alkyl-isoquinolinium bromide, benzalkonium chloride, dodecyl trimethyl ammonium chloride, benzyl dimethyl steryl ammonium chloride and cetyl trimethyl ammonium bromide.

Non-ionic surfactants include, but are not limited to poloxamers, polysorbates, and ethoxylated fatty acids. Poloxamers are block copolymers of ethylene oxide and propolylene oxide and are commercially available, for instance, from BASF under the trade name Pluronic. Polysorbates include polyethoxylated sorbitol esters, which are typically polyethoxylated monoesters. These are commercially available under the trade name Tween from ICI. Other non-ionic surface active agents include polyoxyethylenated alkylphenols, polyoxyethylenated alcohols, fatty acid polyoxyethylenated esters, polyoxyethylenated alkylamines, glycerol esters, polyglycerol esters, tetritol esters, pentritol esters, hexitol esters, anhydrohexitol esters and polyoxyalkylaminated polyol esters. Typically the sufactant is present in the composition in the range of about 0.001 to about 15% by weight of the composition.

The composition may also contain divalent metal cations, such zinc, copper, selenium, calcium or magnesium. These may be in the form of soluble inorganic salts such as zinc chloride or may be in the form of organic or inorganic complexes, such zinc aluminosilcate, zinc carboxymethyloxysuccinate, sodium selenite, cupric gluconate, or other metal complexes known as zeolites. The amount of divalent metallic ion will typically be in the range of 0.001 to 3.0% by weight of the composition.

The compositions may also contain oligosaccharides, such as fructo-oligosaccharides, soy oligosaccharides, inulin, or other oligosaccharides and dietary fiber useful in promoting growth of colonic microflora, for example Lactobacillus and Bifidobacteria. The oligosaccharide will be in a water-soluble form so it may be converted to soluble salt, if necessary. The oligosaccharide, if present, will be present in an amount of at least 0.01 weight percent of the composition.

The compositions of the invention are formulated into commonly utilized dental treatment agents such as mouth washes, oral sprays, oral rinse, toothpaste, gels, dental butter, buccoadhesive tablets, oral films, rapid dissolving tablets and films, chewable polymers and raw hides, and oral swabs and wipes. The method of using the compositions involves treatment of mammals, particularly humans and companion animals, in need of reduction of plaque, or anticaries and antiperidontopathic therapy.

Other additives may be used in the composition, depending upon the method of delivery. Oral rinses may contain acidifying agents, such as malic acid. Mouthwashes and rinses may contain a desensitizing agent, such as, sodium benzoate. Toothpastes may contain polishing agents such as calcium carbonate, sodium bicarbonate, tricalcium phosphate, hydrated alumina, silica, bentonite, dicalcium phosphate; solubiizing agents such as propylene glycol, glycerol, vegetable oil, ethanol; other flavorants such as xylitol; thickeners and adhesive gums such as, sodium carageenan. Surfactants such as, sodium lauryl sulfate may also be utilized.

For dental butters, swabs or buccoadhesive tablets, insoluble waxes may be used, such as, carnuba wax, beeswax, or other vegetable waxes. Thickeners, adhesive and film-forming gums may be added such as carageenan, gum acacia, agar agar, gum ghatti, locust bean gum, guar gum, alginic acid, pectin, carboxymethyl cellulose, hydroxy propyl methyl cellulose, polyacrylic acid copolymers, chitan and chitan derivatives, and microbially produced polysaccharides such as xanthan or pullulan.

Flavorants may optionally be used, such as, peppermint, spearmint, eucalyptus, menthol, carvone, wintergreen, sassafras, prickly ash bark, clove, sage, cinnamon, lemon, lime, grapefruit, orange, and any number of savory flavors produced from protein or yeast fermentation, hydrolysis and digests.

In tablet and film forms, inert wafer excipients may also be included to assist in molding or casting.

As a general maintenance regimen to reduce plaque, without necessarily requiring therapeutic treatment of caries or peridontal disease, the effective amounts and frequency of administration will typically be about from 1 to 3 times daily in the preferred concentrations of ingredients described above. It is recognized that in order to enhance anticaries or antiperiodontopathic effect of the composition, the composition may contain other ingredients for such therapies in the cases of advanced caries or peridontal disease. It is a primary function of the composition of the present invention to provide a preventative regimen to inhibit the growth of microorganisms in the oral cavity that are responsible for plaque and oral malodor.

The following examples are presented by way of illustration and are not intended to limit the invention in any way.

### EXAMPLE 1

A mouthwash was prepared containing the following:

| | Wt. % |
|---|---|
| G. Glabra extract | 0.47 |
| Water | 99.04 |
| Ethanol | 0.30 |
| Inulin | 0.08 |
| Cetyl pyridinium chloride | 0.05 |
| Pluronic ™ P105 | 0.05 |
| Sodium benzoate | 0.01 |

Efficacy of this delivery system was shown as follows:
1. Minimum Inhibitory Concentration (MIC)

High numbers of microorganisms inhabit the oral cavity. Aerobic and anerobic bacteria combine to produce plaque, the soft sticky film adhering to the surface of the teeth. These same bacteria produce sulfur compounds responsible for oral malodor. MIC studies are commonly used to evaluate the potential effectiveness of oral washes and rinses in reducing undesirable bacteria in the oral cavity.

This in vivo study evaluates and compares the minimum inhibitory concentration at which two antimicrobial preparations (Solutions A & B) prevent the growth of oral microorganisms recognized as typical of those responsible for plaque and oral malodor formation. Solution A represents Example 1 (above); solution B, 0.1% chlorhexidine, represents a bench mark for efficacy, in that it is commonly accepted and used by oral care professionals to reduce plaque and malodor causing microbes.

MICs were established using the method of serial dilutions in BHI broth as established by NCCLS (National Committee for Clinical Laboratory Standards). Viable isolates of select strains of oral microbes were obtained commercially and cultured for ongoing use in the study. Serial dilutions of each of the test solutions were added to test tubes containing cultures of each isolate in BHI broth. Tubes were incubated at 37.5 degrees C. for 24 hr. and evaluated for signs of microbial growth (turbidity). Blank tubes of BHI broth, with and without microbial inoculates, were concomitantly run as a control for process sterility and viability. Test results indicate that the MIC of Example 1 is at parity with that of a well known, commercially available plaque and malodor reducing agent, as shown in Table 1, below.

TABLE 1

| | MIC | MIC |
|---|---|---|
| | Solution A | Solution B |
| Microorganism | Example 1 | 0.1% CHX |
| K. pneumonia | 650 ppm | 650 ppm |
| A. viscosus | 320 ppm | 320 ppm |
| S. mutans | 320 ppm | 320 ppm |
| S. sobrinus | 80 ppm | 80 ppm |
| S. sanguinis | 80 ppm | 80 ppm |
| V. atypica | 320 ppm | 160 ppm |

2. Plaque Reduction Study

Dental plaque is the soft, non-mineralized deposit of bacteria located in the adhesive matrix of salivary glucoproteins and extra cellular bacterial polymers. Its composition is quite similar across various mammals. Therefore, plaque reduction studies in dogs are frequently used to mirror efficacy in humans. The following in vitro study evaluated the reduction in the rate of accumulation of plaque by allowing a dilution of Example 1 to flow through a dog's mouth during the drinking process.

Ten, adult, clinically healthy mixed breed dogs were evaluated in a clinical setting under the direction and care of individuals schooled in the practice. Dog colonies were balanced into 2 groups according to base line plaque accumulation index scores obtained through a 7 day plaque accumulation study. The groups were randomly assigned to 1) treatment or 2) control regimens. Dental cleanings were performed on Day 0. All dogs were fed a commercially available dry dog food. Dogs in the control group were provided fresh water daily, ad libitum. Dogs in the treatment group were provided the same source of water, ad libitum, but containing a measured amount of Example 1 liquid. No other foods, treats or snacks were given to either group.

Dental surfaces of 24 teeth in each dog were graded for plaque accumulation on Day 5. Plaque was disclosed with a 2% eosin solution. Plaque levels were evaluated using a modification of the Turesky index system by which both plaque coverage and plaque intensity were recorded. Study results indicate that dogs in the treatment group developed about 20% less plaque than those in the control group.

### EXAMPLE 2

An oral rinse was prepared containing the following:

| | Wt. % |
|---|---|
| Water | 99.9919 |
| Zinc chloride | 0.0023 |
| Ethanol | 0.0022 |
| Inulin | 0.0012 |
| Pluronic ™ F127 | 0.0012 |
| Cetyl pyridinium chloride | 0.011 |
| Glabridine | 0.0001 |
| Malic acid | q.s. |

### EXAMPLE 3

A toothpaste was prepared containing the following:

| | Wt. % |
|---|---|
| Glabridin | 0.001 |
| Water | 26.099 |
| Calcium carbonate | 24.000 |
| Dicalcium phosphate | 23.000 |
| Xylitol | 15.000 |
| Propylene glycol | 3.000 |
| Sodium selenite | 3.000 |
| Sodium lauryl sulfate | 1.900 |
| Sodium carageenan | 1.500 |
| Flavor oil | 1.500 |
| Usnic acid | 1.000 |

### EXAMPLE 4

An oral spray was prepared containing the following:

| | Wt. % |
|---|---|
| Water | 47.84 |
| Ethanol | 47.83 |
| Flavorant | 2.00 |
| Cetyl pyridinium chloride | 1.00 |
| Usnic acid | 1.00 |
| Pluronic ™ F127 | 0.33 |

### EXAMPLE 5

A dental butter was prepared containing the following:

| | Wt. % |
|---|---|
| Waxy base carrier | 92.17 |
| Flavor | 4.02 |
| Cetyl pyridinium chloride | 3.01 |
| Usnic acid | 0.75 |
| Glabridin | 0.05 |

### EXAMPLE 6

A buccoadhesive tablet was prepared containing the following:

| | Wt. % |
|---|---|
| Inulin | 61.87 |
| Flavorant | 5.00 |
| Alginic acid | 4.60 |
| Carnuba wax | 4.60 |
| Carrageenan | 4.60 |
| Pectin | 4.25 |
| G. Glabra extract | 4.40 |
| Cetyl pyridinium chloride | 4.00 |
| Inert wafer excipients | 2.00 |
| Sodium usinate | 1.33 |
| Cupric gluconate | 1.00 |
| Gum acacia | 1.00 |
| Guar gum | 0.50 |
| Locust bean gum | 0.50 |
| Agar agar | 0.25 |
| Gum ghatti | 0.10 |

### EXAMPLE 7

A rapid dissolving oral film was prepared containing the following:

| | Wt. % |
|---|---|
| Pullulan | 55.00 |
| Glycerine | 26.50 |
| Water | 5.00 |
| Xylitol | 4.00 |
| Flavorant | 4.00 |
| Carageenen | 2.50 |
| G. Glabra extract | 1.60 |
| Gum acacia | 1.25 |
| Sodium selenite | 0.15 |

### EXAMPLE 8

An oral chew was prepared containing the following:

| | Wt. % |
|---|---|
| Rawhide | 97.00 |
| Flavorant | 1.25 |
| Prickly Ash Bark extract | 1.30 |
| Lactic acid | 0.20 |
| G. Glabra extract | 0.020 |
| Copper Gluconate (14% Cu) | 0.005 |

### EXAMPLE 9

An oral swab was prepared containing the following:

| | Wt. % |
|---|---|
| Glycerine | 63.00 |
| Flavorant | 31.00 |
| G. Glabra extract | 2.00 |
| Sodium selenite | 2.00 |
| Usnic acid | 2.00 |
| Thickening agents | q.s. |

The invention has been described with respect to various embodiments and examples, but such embodiments and examples are not limitations since one of ordinary skill in the art will be able to employ substitutes and equivalents without departing from the inventive concept. Accordingly, other embodiments are within the scope of the following claims.

1. An aqueous composition for oral use comprising:
a) an anti-odor and anti-plaque water insoluble extract of Glycyrrhiza glabra; and
b) a cationic surfactant.

2. An aqueous composition according to claim 1 having a pH less than about 5.

3. A composition according to claim 1 wherein said extract contains anti-odor and anti-plaque active components selected from the group consisting of glabrins, lycochaleones, glycyrrhizic acid, glycyrrhetinic acid, and other flavonoids and coumarins.

4. (cancelled)

5. (cancelled)

6. A composition according to claim 1 wherein said cationic surfactant comprises a pyridinium compound.

7. A composition according to claim 6 wherein said pyridinium compound comprises cetyl pyridinium chloride.

8-35. (cancelled)

36. A method for reducing plaque formation in tooth enamel, dentine, and soft tissue in the oral cavity comprising the step of contacting said enamel, dentine and tissue within an anti-plaque reducing effective amount of a composition according to claim 1.

37. A composition according to claim 1 further comprising an oligosaccharide.

38. A compostion according to claim 1 or 37 further comprising a divalent metal cation.

39. A composition according to claim 38 wherein said divalent metal cation is selected from the group consisting of copper, zinc, selenium, calcium, and magnesium.

40. A composition according to claim 37 wherein said oligosaccharide is selected from the group consisting of fructo oligosaccharides, soy oligosaccharides, and inulin.
